(19) United States
(12) Patent Application Publication
(10) Pub. No.: US 2013/0190278 A1
(43) Pub. Date: Jul. 25, 2013

(54) Compounds for the Treatment/Prevention of Ocular Inflammatory Diseases

(71) Applicants: Jocelyne Annat, Dijon (FR); Hélène-Céline Huguet, Dijon (FR); Olivier Lacombe, Is Sur Tille (FR); Luc Lebreton, Daix (FR)

(72) Inventors: Jocelyne Annat, Dijon (FR); Hélène-Céline Huguet, Dijon (FR); Olivier Lacombe, Is Sur Tille (FR); Luc Lebreton, Daix (FR)

(73) Assignee: Laboratoires Fournier SA, Dijon (FR)

(21) Appl. No.: 13/813,006

(22) PCT Filed: Jul. 8, 2011
(86) PCT No.: PCT/FR2011/051639

§ 371 Date: Apr. 8, 2013

(30) Foreign Application Priority Data
Jul. 29, 2010 (FR) 1056290
Apr. 1, 2011 (FR) 1152836

(51) Int. Cl. C07C 271/20; A61K 45/06; A61K 31/27

(52) U.S. Cl. CPC C07C 271/20; A61K 31/27; A61K 45/06
USPC 514/171; 514/482

(57) ABSTRACT

The present invention relates to the use of the compounds of formula (I) and their pharmaceutically acceptable salts for the treatment or the prevention of ocular inflammatory diseases, in particular for the treatment and/or prevention of uveitis, severe conjunctivitis, dry eye syndrome or diabetic retinopathy.

### FIELD OF THE INVENTION

The present invention relates to a novel therapeutic use of the compounds of formula (I) as defined below.

More specifically, the present invention relates to the use of these compounds derivatives and their pharmaceutically acceptable salts for the treatment and/or prevention of ocular inflammatory diseases, and more particularly uveitis, severe conjunctivitis (vernal keratoconjunctivitis), dry eye syndrome (keratoconjunctivitis sicca) and diabetic retinopathy.

### BACKGROUND OF THE INVENTION

Ocular inflammatory diseases are the leading cause of visual alteration in the world.

More precisely, uveitis refers to inflammation of the uvea, which is the vascular middle coat of the eye consisting of iris, ciliary body and choroid. Inflammation in uveitis results from a wide variety of traumatic and immune-mediated insults. Conjunctivitis includes diseases characterized by swelling, an itching or burning feeling, or redness of the conjunctiva, which is the membrane covering the white of the eye. The aetiology of conjunctivitis includes infectious and non-infectious conjunctivitis. Conjunctivitis is typically acute in the case of bacterial or viral infections, and chronic in the case of an allergy.

Dry eye syndrome is one of the most common ocular diseases. It is also called keratoconjunctivitis sicca (KCS). It is characterized by symptoms of eye irritation, and can cause blurred vision, these symptoms increasing the risk of corneal infection and ulceration. The pathogenesis of dry eye is not fully understood, although it is widely recognized that dry eye is associated with ocular surface inflammation.

Diabetic retinopathy is a consequence of chronic hyperglycaemia, leading to capillary lesions with functional alterations such as edema and ischemia. Laser photocoagulation is still the standard of care treatment, and vitrectomy is used in case of retinal detachment. Lucentis® (ranibizumab) is used for the treatment of macular edema.

The main therapeutic choice for subjects with uveitis, severe conjunctivitis and dry eye syndrome consists of corticosteroids administered locally or systemically. Nevertheless corticosteroids have severe side effects via the systemic route but also via the local route, such as cortisone-induced cataract or glaucoma, secondary infection and delayed wound healing.

There are also non-steroidal anti-inflammatory agents such as diclofenac or flurbiprofen. However, many of the subjects are not responding or become refractory to steroidal or non-steroidal therapy.

There are also antimetabolite drugs such as azathioprine and methotrexate with hemato- and hepatotoxicity, which are essentially used for the treatment of recalcitrant and very severe uveitis, and immunosuppressants such as cyclosporine A and tacrolimus, administered by oral route, that also show many side effects, such as risks of kidney impairment, an increase of the risk of lymphoproliferative syndromes and malign skin diseases. In order to limit these side effects, these immunosuppressants can be used by the topical route, these compounds are however not soluble in water media due to their macrocyclic structures. They are formulated notably in oil vehicles which have the disadvantage to be irritant, painful and cause blurred vision. These compounds are overall not well tolerated by subjects.

The present invention overcomes the disadvantages from the prior art by providing a novel use of one or more compounds of formula (I) and their pharmaceutically acceptable salts and particularly their use in the treatment and/or prevention of ocular inflammatory diseases such as uveitis, severe onjunctivitis, dry eye syndrome or diabetic retinopathy.

In addition the present invention provides aqueous pharmaceutical compositions, containing the compounds of formula (I), which can reach the posterior chamber of the eye. This represents a huge step forward for the treatment of ocular inflammatory diseases.

Furthermore, the compounds of the present invention have little or no effect on the systemic immune response, which therefore significantly limits the potential side effects associated with the administration of said compounds.

### SUMMARY OF THE INVENTION

The present invention is based on unexpected results demonstrating that the compounds of formula (I) (hereafter referred to as “compounds of the invention”) and their pharmaceutically acceptable salts are able, when administered locally, to improve clinical signs in uveitis models, and especially protect the blood ocular barrier and the ocular tissues of the anterior and posterior chamber without modification of the systemic immune response. The compounds of the invention are likewise useful for the treatment of severe conjunctivitis, dry eye syndrome and diabetic retinopathy.

The beneficial effects of the compounds of the invention and their pharmaceutically acceptable salts, and in particular tresperimus and anisperimus, obtained in different pharmacological models, suggest that these compounds are capable of inducing a regulation of macrophage activation and of the response mediated by T lymphocytes in the eye.

The compounds of the invention and their pharmaceutically acceptable salts, with their linear structure, are soluble and stable in aqueous media therefore they can be locally administered in aqueous formulations which are perfectly biocompatible and which do not cause irritation or blurred vision.

According to a first aspect, the present invention therefore relates to the use of the compounds of the invention and their pharmaceutically acceptable salts, in particular tresperimus and anisperimus, in the preparation of a drug useful for the treatment and/or prevention of ocular inflammatory diseases, in particular uveitis, severe conjunctivitis, dry eye syndrome and diabetic retinopathy.

According to a second aspect, the present invention provides a method of treating and/or preventing ocular inflammatory diseases, notably uveitis, severe conjunctivitis, dry eye syndrome or diabetic retinopathy, comprising administering to a subject in need thereof one or more compounds of the invention or a pharmaceutically acceptable salt thereof. In one embodiment, the compound of the invention or its pharmaceutically acceptable salt is tresperimus or anisperimus. The compound(s) of the invention is (are) administered as eye drops, as a solution which can be injected by the intraocular or the periocular route, or as an implantable system.

The compounds of the invention and their pharmaceutically acceptable salts, in particular tresperimus and anisperimus, are in particular useful for the treatment and/or prevention of uveitis, dry eye syndrome or diabetic retinopathy.

According to a third aspect, the present invention relates to suitable formulations of a pharmaceutical composition comprising as sole active substance a compound of the invention or a pharmaceutically acceptable salt thereof to provide a local administration to subjects with ocular inflammation diseases; the invention more precisely relates to a pharmaceutically acceptable aqueous formulation suitable for local administration.

According to a fourth aspect, the compounds of the invention, in particular tresperimus and anisperimus, and their pharmaceutically acceptable salts, can be administered in combination with an anti-VEGF agent, an anti-TNF agent, a corticosteroid, a non-steroidal anti-inflammatory agent, an antibiotic or an immunosuppressant.

A further understanding of the nature and advantages of the present invention may be made by reference to the remaining portions of the specification and to the claims.

### DESCRIPTION OF DRAWINGS

FIG. 1 shows the effect of a tresperimus injection on clinical Experimental Auto-immune Uveoretinis (EAU) in rat.

FIG. 2 shows the effect of an intravitreal (IVT) tresperimus injection on EAU histopathological scores (A) and EAU histopathological changes in rats treated with tresperimus (C) compared to rats injected with a saline solution (B). a, b, d, e=photoreceptor layers; c, f=optic nerve heads.

FIG. 3 shows the effect of tresperimus in Delayed Type Hypersensitivity (DTH) specific to S-antigen in rats treated by intravitreal injection.

FIG. 4 shows the ocular distribution of tresperimus after instillation of eye drops of a 1% solution twice a day for 4 days in male New Zealand rabbit.

FIG. 5 shows the effect of tresperimus after treatment by instillation on clinical signs of uveitis induced by LPS (lipopolysaccharide).

FIG. 6 shows the effect of tresperimus after treatment by instillation on the number of infiltrating inflammatory cells in uveitis induced by LPS.

FIG. 7 shows the effect of tresperimus on tear volume measured by the Phenol Red test.

FIG. 8 shows the effect of tresperimus on the stability of tear film measured by the time of rupture of the tear film.

FIG. 9 shows the effect of tresperimus on the production of MCP-1 and IL-6 in the vitreous body.

FIG. 10 shows the effect of tresperimus on the amplitude of pseudo-oscillations at different frequencies.

### DETAILED DESCRIPTION

Unless stated otherwise, all technical and scientific terms used herein have the same meaning as commonly understood by those of ordinary skill in the art to which this invention pertains. In addition, the following definitions are provided to assist the reader in the practice of the invention.

The “subject” is preferably a mammal, more preferably a human.

The term “intended to be used in the treatment and/or prevention” as used herein is to be understood as covering the direct use of the compound or salt thereof for the treatment and/or prevention of the specified disease.

“A method of preventing and/or treating” is to be understood as covering the methods wherein a compound or derivative or salt thereof is administered for the treatment and/or prevention of the specified disease.

“Ocular inflammatory diseases” is a general term for inflammation affecting any part of the eye or surrounding tissue. Inflammation involving the eye can range from the familiar allergic hay fever conjunctivitis to rare conditions potentially leading to blindness, such as severe conjunctivitis (vernal keratoconjunctivitis), uveitis, scleritis, episcleritis, optic neuritis, keratitis, orbital pseudotumor, retinal vasculitis, dry eye syndrom, diabetic retinopathy, and age-related macular degeneration (AMD), an ocular manifestation of systemic disease damage to eye tissues, i.e. the retina, which can eventually lead to blindness. The location of the inflammation governs the diagnostic name for the ocular inflammatory disease. Ocular inflammatory diseases can result from several causes.

According to the present invention, uveitis is non-infectious and comes from traumatic causes induced by drugs, from causes with immune mediation, from malignant causes, or from post-ophthalmic surgery causes.

The pharmaceutical compositions of the present invention can also be used after ophthalmic surgery, such as cornea transplantation, causing an ocular inflammation.

“Uveitis” refers to the inflammation of the uvea, the vascular middle coat of the eye comprising the iris, the ciliary body and the choroid. It is classified by its location, its clinical course and its laterality.

“Anterior” refers to iris, cornea, pupil, aqueous humor or ciliary body involvement. For example, Kawasaki disease can be cited as anterior uveitis.

“Intermediate” refers to the vitreous body, pars plana, peripheral retina and sclera.

“Posterior” refers to the choroid or the retina, by extension the fovea and optic nerve. Among non-infectious posterior uveitis, Behcet's disease, Vogt-Koyanagi-Harada disease, pars planitis, sarcoidosis, idiopathic retinal vasculitis and multifocal retinochorioditis can be mentioned.

“Panuveitis” is used when two ore more segments are affected.

According to the present invention, conjunctivitis is non-infectious and mainly comes from serious ocular allergies since it sometimes leads to ulcers which always include a risk of important and definitive visual loss.

Allergic conjunctivitis is an inflammatory reaction of the conjunctiva (a fine membrane covering the eye and the inner part of the eyelid). The eyes can then become red, sting, burn, itch, scratch and weep. Light is difficult to tolerate (photophobia). The eyelids are often red and swollen, and conjunctiva swelling (chemosis), or even a deeper marking of the eyes contours or important mucus secretions, are sometimes noticed. Conjunctivitis hardly affects the cornea. It is the more frequent and probably less serious form of ocular allergy. This type I reaction is often the consequence of abundant pollens during spring- and summertime (tree and grass pollens). The term “allergic keratoconjunctivitis” is used when the damage also concerns the cornea and not only the conjunctiva. There are other types of rarer, more specific but also more serious allergies, which sometimes combine a type I sensitivity with type IV sensitivity. For example vernal conjunctivitis is a serious form of ocular allergy since it sometimes leads to ulcers which always include a risk of important and definitive visual loss. These ulcers are often located in the upper part of the cornea, and papillae form on the conjunctiva notably on the upper eyelid.

Like uveitis, severe conjunctivitis is treated with corticosteroids, non-steroidal anti-inflammatory agents or immunosuppressants.

“Dry eye syndrome or keratoconjunctivitis sicca or ocular dryness” refers to all the pathologies of the eye resulting from the secretion by tear glands of inadequate amount or quality of tears. In the present application dry eye syndrome also concerns all forms of tear deficiency (including autoimmune Sjögren's syndrome and non-Sjögren tear deficiency) and evaporative forms. Dry eye is also known as the disruption of the tear functional unit, which is an integrated system comprising tear glands, the ocular surface (cornea, conjunctiva and meibomian glands) and the eyelids, as well as sensory nerves that connect them.

“Diabetic retinopathy” refers to damage to retinal and choroidal microcirculation (the damaged organs are retina, choroid, papilla and iris) due to chronic hyperglycaemia. Two forms exist: simple (or non-proliferative) and proliferative. In some cases a retinal and generally macular edema appears. In other cases, occlusions of retinal capillaries occur thus causing a retinal ischemia. Moreover, these two main characteristics can combine one with the other thus leading to retinal peripheral ischemia and macular exudates.

The compounds of the invention are also useful for the treatment of age-related macular degeneration (AMD).

The compounds of the invention have the formula (I):

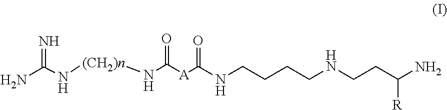

in which:
n is equal to 6 or 8 and,
A is a bond, a group CH₂, a group CH(OH), a group CHF, a group CH(OCH₃), a group CH₂NH or a group CH₂O,
R is an hydrogen atom or a CH₃.

The “salts” of the compounds of the invention can be obtained by chemical reaction between an inorganic or organic acid with the compounds of formula (I) mentioned below.

The preferred inorganic acids for salt formation are: hydrochloric acid, hydrobromic acid, sulfuric acid, phosphoric acid.

The preferred organic acids for salt formation are: fumaric acid, maleic acid, oxalic acid, citric acid, trifluoroacetic acid, tartaric acid and sulfonic acids (from methanesulfonic acid to dodecanesulfonic acid).

The compounds of formula (I) are advantageously chosen from:
N-[4-[(3-aminopropyl)amino]butyl]-carbamic acid, 2-[[6-[(aminoiminomethyl)amino]hexyl]amino]-2-oxoethyl ester;
N-[4-[(3-aminopropyl)amino]butyl]-N′-[6-[(aminoiminomethyl)amino]hexyl]-propanediamide;
N-[4-[(3-aminopropyl)amino]butyl]-N′-[6-[(aminoiminomethyl)amino]hexyl]-2-hydroxy-propanediamide;
N-[4-[(3-aminopropyl)amino]butyl]-N′-[6-[(aminoiminomethyl)amino]hexyl]-2-fluoro-propanediamide;
N-[6-[(aminoiminomethyl)amino]hexyl]-N′[4-[(3-aminopropyl)amino]butyl]-2-methoxy-propanediamide;
N-[6-[(aminoiminomethyl)amino]hexyl]-2-[[[[4-[(3-aminopropyl)amino]-butyl]amino]carbonyl]amino]-acetamide;
N-[6-[(aminoiminomethyl)amino]hexyl]-N′-[4-[(3-aminopropyl)amino]butyl]-ethanediamide;
N-[8-[(aminoiminomethyl)amino]octyl]-N′-[4-[(3-aminopropyl)amino]butyl]-ethanediamide;
N-[8-[(aminoiminomethyl)amino]octyl]-N′-[4-[(3-aminopropyl)amino]butyl]-propanediamide;
N-[8-[(aminoiminomethyl)amino]octyl]-N′-[4-[(3-aminopropyl)amino]butyl]-2-hydroxy-propanediamide;
N-[8-[(aminoiminomethyl)amino]octyl]-N′-[4-[(3-aminopropyl)amino]butyl]-2-fluoro-propanediamide;
N-[4-[(3-aminopropyl)amino]butyl]-2-methoxy-N′-[8-[(aminoiminomethyl)amino]-octyl]-propanediamide;
N-[8-[(aminoiminomethyl)amino]octyl]-2-[[[[4-[(3-aminopropyl)amino]butyl]-amino]carbonyl]amino]-acetamide;
N-[4-[(3-aminopropyl)amino]butyl]-carbamic acid, 2-[[8-[(aminoiminomethyl)-amino]octyl]amino]-2-oxoethyl ester;
N-[4-[(3-aminobutyl)amino]butyl]-carbamic acid, 2-[[6-[(aminoiminomethyl)amino]-hexyl]amino]-2-oxoethyl ester;
N-[4-[(3-aminobutyl)amino]butyl]-N′-[6-[(aminoiminomethyl)amino]hexyl]-ethanediamide;
N-[4-[(3-aminobutyl)amino]butyl]-N′-[6-[(aminoiminomethyl)amino]hexyl]-propanediamide;
2-[[[[4-[(3-aminobutyl)amino]butyl]amino]carbonyl]amino]-N-[6-[(aminoiminomethyl)amino]hexyl]-acetamide;
N-[4-[(3-aminobutyl)amino]butyl]-N′-[6-[(aminoiminomethyl)amino]hexyl]-2-hydroxy-propanediamide;
N-[4-[(3-aminobutyl)amino]butyl]-N′-[6-[(aminoiminomethyl)amino]hexyl]-2-fluoro-propanediamide;
N-[4-[(3-aminobutyl)amino]butyl]-N′-[6-[(aminoiminomethyl)amino]hexyl]-2-methoxy-propanediamide;
N-[4-[(3-aminobutyl)amino]butyl]-N′-[8-[(aminoiminomethyl)amino]octyl]-ethanediamide;
N-[4-[(3-aminobutyl)amino]butyl]-N′-[8-[(aminoiminomethyl)amino]octyl]-propanediamide;
N-[4-[(3-aminobutyl)amino]butyl]-carbamic acid, 2-[[8-[(aminoiminomethyl)-amino]octyl]amino]-2-oxoethyl ester;
2-[[[[4-[(3-aminobutyl)amino]butyl]amino]carbonyl]amino]-N-[8-[(aminoiminomethyl)amino]octyl]-acetamide;
N-[4-[(3-aminobutyl)amino]butyl]-N′-[8-[(aminoiminomethyl)amino]octyl]-2-hydroxy-propanediamide;
N-[4-[(3-aminobutyl)amino]butyl]-N′-[8-[(aminoiminomethyl)amino]octyl]-2-fluoro-propanediamide;
N-[4-[(3-aminobutyl)amino]butyl]-N′-[8-[(aminoiminomethyl)amino]octyl]-2-methoxy-propanediamide;
   and the pharmaceutically acceptable salts thereof.

Preferred compounds of formula (I) are chosen from:
N-[4-[(3-aminopropyl)amino]butyl]-carbamic acid, 2-[[6-[(aminoiminomethyl)-amino]hexyl]amino]-2-oxoethyl ester (tresperimus) and
N-[4-[(3-aminobutyl)amino]butyl]-carbamic acid, 2-[[6-[(aminoiminomethyl)-amino]hexyl]amino]-2-oxoethyl ester,
   and the pharmaceutically acceptable salts thereof.

Especially preferred compounds of formula (I) are N-[4-[(3-aminopropyl)amino]butyl]-carbamic acid, 2-[[6-[(aminoiminomethyl)amino]hexyl]-amino]-2-oxoethyl ester, tris-hydrochloride and N-[4-[(3-aminobutyl)amino]butyl]-carbamic acid, 2-[[6-[(aminoiminomethyl)amino]hexyl]amino]-2-oxoethyl ester, tetra-hydrochloride.

The pharmaceutical compositions of the invention typically comprise a compound of the invention or a pharmaceutically acceptable salt thereof as sole active substance, together with one or more pharmaceutically acceptable carriers or excipients.

“Pharmaceutical carriers” refer to a pharmaceutically acceptable excipient or a mixture of several pharmaceutically acceptable excipients which enable the administration of active substances. They enable and can facilitate or improve the preparation of the composition and can stabilize the composition. Moreover, pharmaceutically acceptable carriers can enhance the composition efficacy, improve ocular tolerability of the active substance and/or modify its release profile.

They must also be both pharmaceutically and physiologically acceptable in the sense of being compatible with the other ingredients of the composition, and biocompatible and non-toxic. Such carriers may take a wide variety of forms depending on the form of preparation desired for administration, e.g. local administration.

“Local administration” is to be understood as defining all ocular routes i.e. topical and injectable administration, and administration by means of implantable systems.

“Topical administration” can be in the form of for example, and in a non-limiting way, eye drops, collyrium or ocular instillation, sprays, creams, ointments, gels, hydrogels, oleogels, hydrophilic lens, inserts, and implants. Dosage forms can be for example, and in a non-limiting way, solutions, suspensions, colloidal systems (e.g. liposomes, emulsions, microemulsions, nanoemulsions, microparticles, nanoparticles, microspheres, niosomes, dendrimers), micelles, mixed micelles, complexing systems e.g. cyclodextrin solutions, as well as non implantable inserts in the form of for example, and in a non-limiting way, discs, films or strips.

“Injectable administration” can be in a non-limiting way intraocular (intravitreal, IVT), periocular including subconjunctival, sub tenon's, retrobulbar and intrascleral administration.

“Intravitreal administration” can be carried out as injectable or implantable systems. Dosage forms can be in a non-limiting way solutions, suspensions, colloidal systems (e.g. liposomes, emulsions, microemulsions, nanoemulsions, microparticles, nanoparticles, microspheres, niosomes, dendrimers), micelles, mixed micelles, as well as biodegradable or non-biodegradable implants in the form of for example, and in a non-limiting way, rods, nails, pellets.

In the present application when a concentration is expressed in m/V it is considered that the density of the solution is 1.

For both administration routes (topical and injectable), depending of the compound to be delivered, most of the dosage forms cited above can potentially increase the residence time of the active principle at the surface of the eye or in the vitreous body, provide a slow and sustained release of encapsulated compounds, and/or avoid toxicity and increase ocular tolerability.

According to the present invention, for the treatment and/or prevention of ocular inflammatory diseases, and in particular uveitis, severe conjunctivitis, dry eye syndrome or diabetic retinopathy, the compounds of the invention or their pharmaceutical acceptable salts can be administered via an aqueous pharmaceutically acceptable composition or formulation suitable for topical administration, preferably by instillation, or for injectable administration, preferably an intravitreal administration.

For topical or injectable administration, the excipient(s) must be pharmaceutically acceptable and suitable for this type of ocular administration.

The aqueous media used in the present invention consist of water that does not contain physiologically and ophthalmologically adverse agents. The pharmaceutical composition of the invention is in the form of an aqueous formulation with a pH physiologically compatible for the ocular route. “pH physiologically compatible for the ocular route” is intended to mean a pH in the range from about 5.5 to about 8, preferably from about 6.0 to about 7.5. The pH of the preparations is adjusted with an acid such as for example acetic acid, boric acid, lactic acid, hydrochloric acid; a base such as for example sodium hydroxide, sodium borate, sodium citrate, sodium acetate; or a pharmaceutically acceptable buffered solution such as for example sodium phosphate buffer, potassium phosphate buffer, sodium citrate buffer. The aqueous preparations of the invention are isotonic and physiologically adapted for ocular, topical and intraocular administration. The osmotic pressure of the preparations is close to physiological pressure and is generally comprised between about 200 mOsm and about 400 mOsm, preferably between about 260 and about 340 mOsm. If necessary, the osmotic pressure can be adjusted using suitable amounts of physiologically and ophthalmologically acceptable excipients. Sodium chloride is usually used as a tonicity agent at a concentration (expressed in m/V) not exceeding 0.9%. Equivalent amounts of one or more salts comprised of a cation and an anion can also be used. Depending on the therapeutic indication of the present invention, the osmotic pressure can optionally be corrected by adding sugars or polyols, alone or as a combined mixture. The preparations of the present invention have a viscosity varying from 0 to about 2000 Centipoises, preferably lower than about 100 Centipoises, and more preferably lower than about 30 Centipoises.

The composition of the present invention can contain agents increasing the viscosity thereby extending the precorneal dwelling time of the active principle after instillation. These viscosifying agents can also have mucoadhesive properties. Mucoadhesive polymers capable of creating non covalent bonds with glycoproteins which are notably present in the conjunctiva can be used in the present invention to locally limit the formulation to the eye, to optimize the dwelling time of the formulation locally and potentially increase the ocular bioavailability of the active principle, and to reduce the administration frequency thereby improving therapeutic compliance. These polymers are usually macromolecular hydrocolloids. They can be used alone or in combination in the present invention and are for example cellulose derivatives such as methylcelluloses, sodium carboxymethylcelluloses, hydroxyethylcelluloses, hydroxypropylcelluloses, hydroxypropylmethylcelluloses; acrylic derivatives such as for example salts of polyacrylic acid and its functionalized derivatives (or polycarbophils); carbomers; natural products such as for example alginates, chitosans, pectins, hyaluronic acid and its derivatives; polysaccharide derivatives such as for example gellan gum and its derivatives, xanthan gum, carrageenans; co-polymers such as poloxamers. Polymers having an in situ gelling capacity can be incorporated in the preparation of the pharmaceutical composition of the invention. The so-called phase transition systems are liquid and lead to the formation of gels compatible with the ocular function by ionic activation depending on the pH and temperature. For example, polymers such polyacrylic acid derivatives, cellulose derivatives, methylcelluloses, copolymers and poloxamers can be cited.

The composition of the present invention can also contain excipients well-known to the skilled person, for example surfactants, co-surfactants, co-solvents, penetration agents, gelling agents, emulsifiers, antioxidants, preservatives, polymers for sustained release.

The pharmaceutical composition can also be in the form of an insert or a solid implant which enables an ocular administration and a sustained release of the active principle. For example the preparation of inserts can be carried out using a water-soluble solid polymer. Inert polymers biocompatible for the ocular route, which are used for the preparation of an insert suitable for the ocular route, are synthetic, semi-synthetic or of natural origin. The composition of solid implants can also consist of synthetic, semi-synthetic or natural polymers, preferably biodegradable polymers such as for example polyvinyl alcohols, polylactic-co-glycolic acids, poly-epsilon caprolactones, hyaluronic acid esters. These biodegradable polymers can also be used to encapsulate the active principle in microspheres, nanospheres or nanocapsules dispersed in aqueous solution to provide a sustained and targeted release of the active principle.

Other matrices such as water-soluble lenses impregnated with or containing the active principle can increase the dwelling time of the active principle at the surface of the eye.

The principles for manufacturing and sterilizing these formulations are conventionally well-known in the field of dosage form techniques.

According to a first preferred embodiment, the pharmaceutical composition in the form of eye drops or injectable solution comprises an effective dose of a compound of the invention such as for example tresperimus, dissolved in a physiological aqueous solution as main carrier. This solution ideally comprises an aqueous sodium chloride solution at a concentration preferably not greater than 0.9% (m/V), or an aqueous glycerol solution at a concentration preferably not greater than 2.5% (m/V) in order to obtain a tonicity of the pharmaceutical composition comprised between about 260 and about 340 mOsm. This solution is adjusted to a pH close to 6.5 with, for example, sodium hydroxide. A bioadhesive polymer, such as preferably hyaluronic acid or a derivative thereof, is added. This dosage form is sterilized preferably by gamma radiation. This dosage form can be in the form of unidose packs.

According to another preferred embodiment, the injectable solution for administration as a biodegradable implant comprises an effective dose of at least a compound of the invention encapsulated preferably in micro- or nanoparticles made of poly-epsilon-caprolactones.

A major advantage of the present invention is that all ocular tissues (anterior or posterior chambers) are exposed to the compounds of the invention, for example upon administration of a pharmaceutically acceptable aqueous composition. In a study evaluating the ocular distribution of tresperimus in male New Zealand rabbits (FIG. 4) after eye drop instillation of a 1% solution twice a day for four days, it was noticed that the retina/choroid (posterior chamber) and the ciliary body/iris (anterior chamber) were exposed to tresperimus levels from 0.5 to 0.7 μM and 0.3 to 0.7 μM over 24 hours after repeated eye drop instillation twice a day as a simple 1% aqueous solution. Therefore in comparison to another local administration such as intraocular injection or implants, and in addition to a better compliance, topical administration of the compounds of the invention can allow a much better control of active substance concentrations in ocular tissues. Moreover, plasma levels were observed to be low (<50 ng/mL from 5 min to 2 h post-instillation) and during a short period of time below the lower limit of quantification (Blq) (2 ng/mL 4 h post-dose) after eye drop instillation. This allows avoiding undesired immunosuppressive systemic effects.

The concentration of the therapeutically active substance in the formulations for the intravitreal route can vary from 0.1 μM to 100 mM, preferably from 1 μM to 10 mM, and more preferably from 10 μM to 0.1 mM. The concentration of the therapeutically active substance in the formulations for ocular topical instillation can vary from 0.001% to 5% (expressed as m/V), preferably from 0.001% to 1.5%, more preferably from 0.01% to 1.5%. These concentrations can be applied for other ocular local administration routes and can vary depending on the therapeutic indication. The route of administration and the dose will be left to the discretion of the physician depending on the subject, his symptoms and the severity of his disease.

These compositions are prepared by any process for manufacturing dosage forms well-known in the field of pharmaceutical techniques.

According to another aspect, the compound(s) of the present invention can be combined with or used in combination with other therapeutic agents. For example, a subject can be treated with one or more compounds of the invention or a pharmaceutically acceptable salt thereof, in particular tresperimus and/or anisperimus, along with other conventional drugs for the treatment of inflammatory ocular diseases. The various active substances can be administered simultaneously, sequentially or over a period of time. The compound of the invention or pharmaceutically acceptable salt thereof will preferably not be administered in combination with a Lck enzyme inhibitor.

According to an embodiment, the present invention thus relates to a pharmaceutical composition comprising, as active substances, at least one compound of formula (I) or a pharmaceutically acceptable salt thereof in combination with one or more drugs used in the treatment of uveitis, selected from corticoids such as for example dexamethasone, prednisolone and triamcinolone; immunosuppressants having a mechanism of action different from that of the compounds of the invention such as, for example, cyclophosphamide, methotrexate, azathioprine, cyclosporine A, tacrolimus, sirolimus, mycophenolate mofetil; anti-TNF agents such as, for example, rituximab, daclizumab, infliximab, adalimumab and etanercept.

According to an embodiment, the present invention thus relates to a pharmaceutical composition comprising, as active substances, at least one compound of formula (I) or a pharmaceutically acceptable salt thereof in combination with one or more drugs used in the treatment of severe conjunctivitis, selected from corticoids such as, for example, dexamethasone, prednisolone; non-steroidal anti-inflammatory agents such as nedocromil, liodoxamide, olopatadine; antibiotics, antifungals and antibacterials such as tobramycine, natamycine, moxifloxacine; immunosuppressants having a mechanism of action different from that of the compounds of the invention such as, for example, cyclosporine A, tacrolimus, sirolimus.

According to another embodiment, the present invention relates to a pharmaceutical composition comprising, as active substances, at least one compound of the invention or a pharmaceutically acceptable salt thereof in combination with one or more drugs used in the treatment of dry eye syndrome, selected from immunosuppressants having a mechanism of action different from that of the compounds of the invention such as, for example, cyclosporine A and mycophenolate mofetil; corticosteroids such as, for example, loteprednol, rimoxelone and fluorometholone; and tetracyclines. They can also be used in combination with artificial tears and secretogogues.

According to another embodiment, the present invention relates to a pharmaceutical composition comprising, as active substances, at least one compound of the invention or a pharmaceutically acceptable salt thereof in combination with one or more drugs used in the treatment of diabetic retinopathy, selected from anti-VEGF agents such as, for example, ranibizumab, pegatapnib, bevacizumab; anti-TNF agents such as, for example, rituximab, daclizumab, infliximab, adalimumab and etanercept; corticosteroids such as, for example, dexamethasone, prednisolone and triamcinolone; and immunosuppressants having a mechanism of action different from that of the compounds of the invention such as, for example, cyclosporine A, tacrolimus, everolimus and sirolimus. They can also be used in combination with laser therapy (photocoagulation).

The invention will be illustrated in more detail in the examples below with reference to tresperimus but the skilled person will appreciate that the present invention is not limited to this compound of formula (I).

It has to be understood that the examples and embodiments described herein are intended only to illustrate the invention and that various modifications or changes made in the light of said examples and embodiments will be suggested to the skilled person and must be included within the spirit and scope of this application and appended claims. Although methods and material similar to those described herein can be used in practice or in the tests of the present invention, preferred methods and materials are described.

### Example 1

### Uveitis

The eye is a site of immunological privilege; however eye diseases originating from an imbalance of the immune system develop and are responsible for vision impairments that can lead to blindness. Animal models, mainly experimental autoimmune uveitis (EAU) and endotoxin-induced uveitis (EIU), are considered as relevant clinical models of ocular diseases and are precious tools to study immunological mechanisms enabling regulation of diseases in man:
EAU induced in rats by immunization with purified retina antigens, mainly S-antigen (S-Ag), is considered as a relevant clinical model for studying the mechanisms of posterior uveitis in man and to develop new therapeutic strategies for uveitis;
EIU is a model of spontaneously resolvent, acute inflammatory uveitis, involving components of the natural immune system. This is a useful model for studying local aspects of ocular inflammation, and is considered as a relevant model of anterior uveitis in man.

In the present invention, we have shown for the first time that local ocular administration of compounds of formula (I) and their pharmaceutically salts is of great benefit in these two experimental models which are considered as relevant clinical models of uveitis in man.

### The EAU Model

EAU models help understand physiopathological mechanisms and in particular the involvement of CD4+ (Cluster of Differentiation 4) lymphocytes, of macrophages and pro-inflammatory cytokines in the mechanisms of retina destruction.

EAU is an inflammatory disease model that shares many clinical and histopathological features with human uveitis, such as sympathetic ophthalmia, birdshot retinochoroidopathy, Vogt-Koyanagi-Harada syndrome, Behcet's disease and sarcoidosis. It is a clinically relevant model for human ocular inflammation.

EAU is induced by immunization with the purified retinal autoantigen, S-antigen (S-Ag) that is also recognized by subjects with uveitis. EAU is dependent on CD4+ Th1 (interferon-gamma producing cells) and CD4+ Th17 (interleukin-17 producing cells) effector cells, each effector phenotype can induce a pathological reaction.

However, IL-17 (interleukin-17) plays a dominant role in EAU induced by the IRBP protein (“interphotoreceptor retinoid-binding protein). Neutralization of IL-17 prevents the disease or reverses its progression. In addition Th17 effector cells induce EAU in the absence of interferon (IFN)-gamma.

Then, macrophages and microglial cells locally amplify the reaction and induce the destruction of photoreceptors and of the retinal tissue. Monocytes/macrophages as well as neutrophils are important effector cells in EAU whereas T-cells are acting more to initiate and maintain the response. Macrophages cross the blood-retina barrier and infiltrate the retina, where the release of mediators such as NO (nitric oxide) and TNF (tumor necrosis factor) can cause severe retinal damage and consequently a loss of vision in subjects. We have studied the effect of local administration of tresperimus on EAU and on the ocular and systemic immune responses induced by S-Ag immunization.

### Materials and Methods

### 1. Induction of EAU in Lewis Rats

Eight-week-old female Lewis rats (R. Janvier, Le Genest Saint Isle, France) were immunized systemically with 40 μg of the retinal autoantigen S-Antigen (S-Ag) purified as previously described (de Kozak Y, Sainte-Laudy J, Benveniste J and Faure J P. Eur J. Immunol. 1981; 11:612-617).

### II. Treatment Protocol

The administration of tresperimus was performed by intravitreal (IVT) injections (5 μL) in both eyes, on days 6, 9 and 12 after S-Ag immunization. At the end of the experiments, i.e. 19-20 days after immunization, rats were anesthetized by intraperitoneal injection of pentobarbital (Sanofi-Aventis, France) before blood collection by intracardiac puncture. Rats were then euthanized with a lethal dose of pentobarbital and both eyes and blood samples were collected for analysis.

In a first experiment, a group of rats received substantially isoosmolar and physiological sterile saline containing 9 mM tresperimus to achieve a 1 mM final solution in the vitreous body, a control group of rats received a vehicle (saline), and a control group of rats was not treated. Animals were examined clinically with a slit lamp from day 9 after S-Ag immunization up to the time of euthanasia. Histopathology of the eyes was performed and immunostaining was processed on sections obtained with a cryostat. Inguinal lymph nodes were taken for RT-PCR analysis of cytokines.

In a second experiment, a group of rats received three injections of tresperimus into the vitreous body and control rats were injected with saline. The rats were observed clinically and subjected to Delayed Type Hypersensitivity (DTH) analysis. Tresperimus levels in plasma and in ocular tissues were measured 1 h, 3 days and 8 days after the third injection.

### III. Evaluation of EAU Severity

1. Clinical Evaluation

Animals were examined with a slit lamp on day 7, and then each day from day 11 up to the time of euthanasia to evaluate the onset time and the severity of the disease. The intensity of the clinical ocular inflammation was scored on a scale from 0 to 7 for each eye as previously described (de Kozak Eur J 1 mm 2004).

2. Histopatholoqy

At the time of euthanasia (day 19-20 after immunization), enucleated rat eyes were fixed, processed, paraffin sections cut and stained with haematoxylin-eosin-safran for histological evaluation. Sections were examined and scored according to the severity of EAU on a semi quantitative scale from 0 to 7 as follows: (0) no tissue destruction, (1-2) destruction of outer segments of rods and cones, (3-4) destruction of the outer nuclear layer, (5-6) destruction of the inner nuclear layer, and (7) destruction of the ganglion cell layer.

3. Immunohistochemistry

Eyes (2 eyes/group) were collected, cryostat sections cut (10 μm) and stained for immunochemistry as described previously on day 19-20 after immunization. The following antibodies were used: an anti-NOS-2 primary antibody (Beckton Dickinson Biosciences, Transduction laboratories, San Jose, USA); an anti-NF-κB/p65 primary antibody, then a secondary antibody conjugated with Alexa Fluor® 488 (Molecular Probes, Eugene, Oreg.); anti-macrosialin CD68 primary antibody (clone ED1) (Serotec, Oxford, GB), then a secondary antibody conjugated to Alexa 564 (red). Sections were observed by fluorescence photomicroscopy (FXA; Microphot; Nikon, Melville, N.Y.) and digitized micrographs were obtained with a digital camera (Spot; BFI Optilas, Evry, France).

### IV. Immune Response Evaluation

1. Delayed Type Hypersensitivity

DTH was estimated by an ear assay measuring the specific anti-S-Ag response 18 days after immunization. Rats were sensitized with 10 μg of S-Ag in the right ear and with saline in the left ear. Specific ear swelling was measured 24 and 48 h after sensitization and the difference in thickness (mm) between the two ears was calculated.

2. RNA Isolation, Reverse Transcription PCR in Lymph Nodes and in Ocular Cells

Total RNA was isolated from lymph nodes draining the immunizing site, 19-20 days after immunization, and from cells collected after centrifugation of aqueous humor/vitreous body from eyes of each group.

### V. Statistical Analysis

Data are presented as mean±Standard Error of the Mean (SEM). EAU and DTH clinical and histological evaluations are compared using the non-parametric Mann-Whitney U test followed by the Bonferroni multiple comparison test. A p-value adjusted by the multiple comparison tests was calculated in each experiment.

### VI. Results

1. Pharmacokinetics of Tresperimus in Ocular Tissues and Plasma after Intravitreal Injections

Tresperimus concentrations in plasma, aqueous humor/vitreous body and the retina/choroid after intravitreal injections of tresperimus in Lewis rats are reported in Table 1:

TABLE 1
Effect of an intravitreal injection of tresperimus on EAU histopathology
on day 19-20 after S—Ag immunization (M ± SEM)
Tresperimus concentrations
(N = 6)

| | | Immunized rats | | |
|---|---|---|---|---|
| | | (3 IVT injections) | | |
| Time post-injection | | 1 h | 3 days | 8 days |
| aqueous humor/vitreous | Mean | 270 | 2.2 | 1.8 |
| (μM) | SEM | 61 | 1 | 1 |
| retina/choroid | Mean | 155 | 36 | 11 |
| (μM) | SEM | 25 | 4 | 4 |
| plasma | Mean | 0.11 | blq | blq |
| (μM) | SEM | 0.03 | — | — |

Blq: below the lower limit of quantification (6 ng/mL)

After intravitreal injection of tresperimus, plasma levels of the test sample were quantified only at the first time point 1 h post-injection, with low mean concentrations around 0.1 μM (about 40 ng/mL). Ocular tissues were highly exposed to tresperimus, with significant contents (>10 μM) in the retina/choroid 8 days post-injection.

2. Intravitreal Injection of Tresperimus is an Effective Treatment of EAU; Clinical Observation

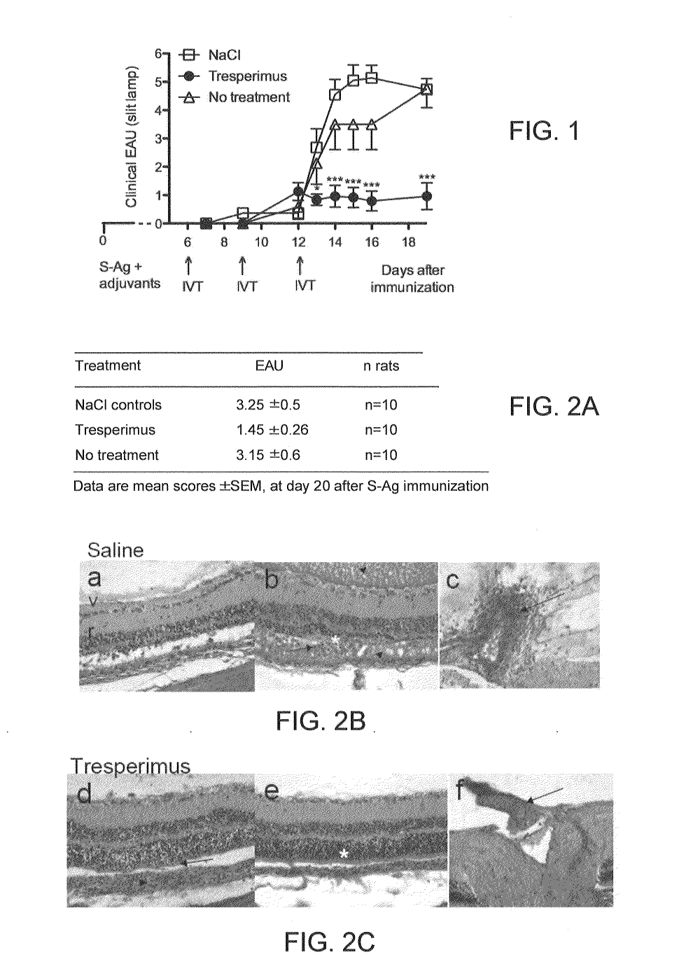

Treatment with tresperimus led to a significant reduction of the clinical severity of EAU from day 13 after immunization compared to rats that received injections of saline (day 13: * p<0.02; days 14 to 19: *** p<0.0006), or compared to rats that did not receive any intraocular treatment (day 12: * p<0.02; day 19: *** p<0.0006) (FIG. 1). The disease severity was significantly reduced by the treatment up to 19 days after immunization, indicating that intraocular therapy is very effective.

3. Intraocular Injection of Tresperimus Protects the Retina from Destruction and Modulates Macrophage Activity

Rats treated with 3 injections of tresperimus presented a very low grade histological EAU (mean EAU severity grade: 1.45±0.26, n=10, p=0.007) compared to lesions observed in control rats injected with saline (mean EAU severity grade: 3.25±0.5, n=10) (FIG. 2A) and compared to rats that did not receive any intraocular treatment (mean EAU severity grade: 3.15±0.6, n=10, p=0.08). The mean EAU histopathological score was based on retina alterations. Histopathological examination of the retinas from control rats injected with saline (FIG. 2B) showed severe posterior uveitis with extensive destruction of the photoreceptor cell layer (a, b, white asterisks), infiltration of the subretinal space by inflammatory cells (arrow) and fibrin exudates in the vitreous body (arrowhead). Numerous inflammatory cells were present in the vitreous body at the optic nerve head level (arrow) (c). In contrast, in rats treated with tresperimus (FIG. 2C), the photoreceptor cell layer was largely spared from destruction (e, white asterisks) or showed partial loss of the outer segments (d, arrow) with an infiltration of the choroid by inflammatory cells (d, arrowhead). No inflammation was visible at the optic nerve head level (f, arrow).

As shown by immunostaining in control rats injected with saline, numerous ED1-positive macrophages and lymphocytes expressed cytoplasmic and nuclear expression of NF-kappaBp65 mainly in the vitreous body where numerous infiltrations by inflammatory cells are visible. In contrast, in tresperimus treated rats, few infiltrations by inflammatory cell are visible in ocular tissues, with a reduced number of infiltrated cells in ocular tissues and media and showing only a cytoplasmic expression of NF-kappaBp65.

4. Intravitreal Injection of Tresperimus has No Effect on Systemic Immune Response In Vivo

a) Cytokines in Inguinal Lymph Nodes (RT-PCR)

No difference in levels of TNF-alpha, IL-2, IFN-gamma and IL-17 was detected in inguinal lymph nodes from treated and control rats indicating that the treatment has no systemic effect.

b) Delayed Type Hypersensitivity

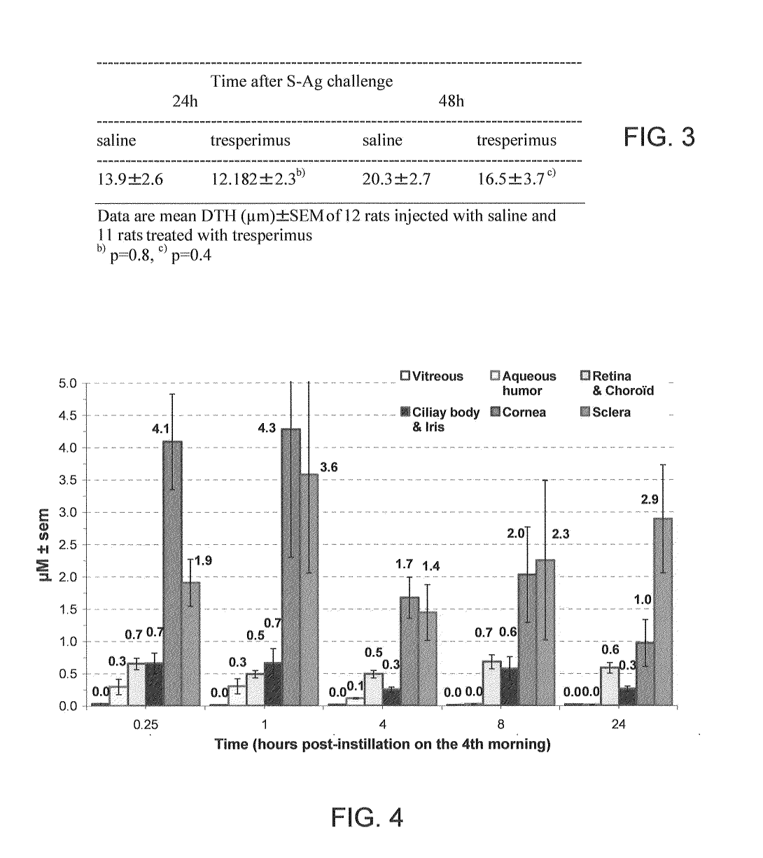

DTH was estimated by an ear assay measuring the specific anti-S-Ag response. Rats treated with tresperimus did not exhibit a significant reduction of ear swelling at 24 h and 48 h compared to control rats that received an IVT injection of saline (p=0.8; p=0.4 respectively) demonstrating that T-cell reactivity towards S-Ag in vivo is not reduced by treatment with tresperimus and confirming that the treatment has no systemic effect (FIG. 3).

In conclusion, injection of tresperimus in the posterior pole of the eye, in the posterior zone of the ciliary body, enabled its diffusion in the anterior and posterior segments of the eye as shown by its efficacy on the anterior and posterior ocular inflammation in EAU. Moreover, low levels (<90 ng/mL) of tresperimus were found in the plasma without any effect on the immune system response. In fact, the effect of tresperimus was limited to the eye, which confirms that no effective diffusion took place in the general circulation. We have shown that three intravitreal injections of tresperimus after immunization with S-Ag during the afferent phase of the disease (days 6, 9, 12) are effective to reduce the clinical ocular inflammation and protect the retinal photoreceptors.

To examine at which level tresperimus acts, delayed type hypersensitivity (DTH) to S-Ag, as assessed by an ear test, was not different in control rats and treated rats (FIG. 3), suggesting that the treatment did not modify the reactivity of systemic T-cells to S-Ag.

Moreover, we have shown that the ocular treatment has no effect on the systemic immune response. In fact, in inguinal lymph nodes draining the immunization site, the level of inflammatory cytokines such as TNF-alpha, and of cytokines produced by T lymphocytes such as IL-2, IFN-gamma (interferon-gamma) and IL-17, was not modified by the treatment with tresperimus.

### The EIU Model

The endotoxin-induced uveitis model is a model of acute ocular inflammation in rats or mice, induced by systemic or local injection of lipopolysaccharide (LPS) of Gram-negative bacteria. This is a model for human acute anterior uveitis which is often associated with systemic disorders, such as during Crohn's disease, ankylosing spondylitis and Blau syndrome.

EIU is characterized by the rupture of the ocular brain barrier, the intraocular infiltration of inflammatory cells into the posterior and anterior segments of the eye, and the production of NO and of inflammatory cytokines and chemokines by infiltrated inflammatory cells, mainly macrophages and polymorphonuclear leukocytes (PMNs), and by ocular cells of the vascular endothelium, of the retinal pigment epithelium, of microglia and of Müller's cells. Although this inflammatory uveitis spontaneously resolves in a few days upon involvement of the natural immune system, it is a source of important lesions of the ocular tissues.

We have tested the effect of tresperimus in this EIU modal in rats after instillation of drops at different concentrations.

### Materials and Methods

### I. Induction of Uveitis by Endotoxin

Eight-week-old female Lewis rats (R. Janvier, Le Genest Saint Isle, France) weighing about 250 g were used in this study and were injected, in the pad of one of their paws, with 200 μg of LPS of Salmonella typhimurium (Sigma) in 0.1 mL sterile water.

### II. Treatment Protocol

Tresperimus was administered by instillation in each eye twice a day for 4 days, of drops at 5% (m/m) and 0.5% (m/m) in a 0.1% (m/m) aqueous solution of sodium hyaluronate.

On the third day LPS was administered in the pad of a paw and 24 h later tresperimus was administered one last time. The animals were then examined with a slit lamp, their blood was collected, and they were sacrificed. The eyes were then collected to be analysed.

### III. Clinical Examination

Animals were examined with a slit lamp 24 h after LPS administration corresponding to the peak of uveitis clinical inflammation. The intensity of inflammation was scored on a scale from 1 to 6 for each eye as previously described (De Kozak Y. et al., J. Neuroimmunol. 1998; 86(2):171-181) and as follows: 0, no sign of inflammation; 1, discrete inflammation of the iris and the conjunctiva; 2, dilation of the iris and the vessels of the conjunctiva; 3, hyperemia in the iris associated with the Tyndall effect in the anterior chamber; 4-6, signs similar to grade 3 but in addition with the presence of a synechia, of a fibrinoid exudation or of a hypopyon. The clinical EIU is considered positive if the grade is equal to or greater than 1.

### IV. Histopathology and Counting of Inflammatory Cells

After the animals were euthanized (i.e. 24 h after LPS injection), rat eyes were enucleated, then fixed and processed. Paraffin sections were cut for histological evaluation. Infiltrated inflammatory cells were counted on the sections made on the anterior segment of the eye (5 sections per eye) after staining with haematoxylin-eosin-safran for histological evaluation. The number of cells is expressed as mean±SEM of the total number of cells in each eye and for each animal as described previously (de Kozak Y. et al, IOVS 1999 September; 40(10):2275-82).

### V. Statistical Analysis

Results are presented as mean±SEM and compared using the Mann-Whitney U test. P<0.05 is considered as statistically significant.

### VI. Results

The effect of tresperimus was evaluated in the EIU model in rats. Acute and bilateral ocular inflammation induced by LPS injection is characterized by the infiltration of inflammatory cells 4 h after the injection. It reaches a maximum between 18 h and 24 h and disappears after 4 days.

Tresperimus was administered by instillation twice a day for 4 days at 5% (m/m) and 0.5% (m/m) in a 0.1% aqueous sodium hyaluronate solution. The results (FIG. 5) are expressed as clinical scores ±SEM for each eye.

In comparison with control animals, the treatment with tresperimus allowed a significant reduction of the ocular inflammation to be obtained (p=0.001 and p=0.0001, respectively).

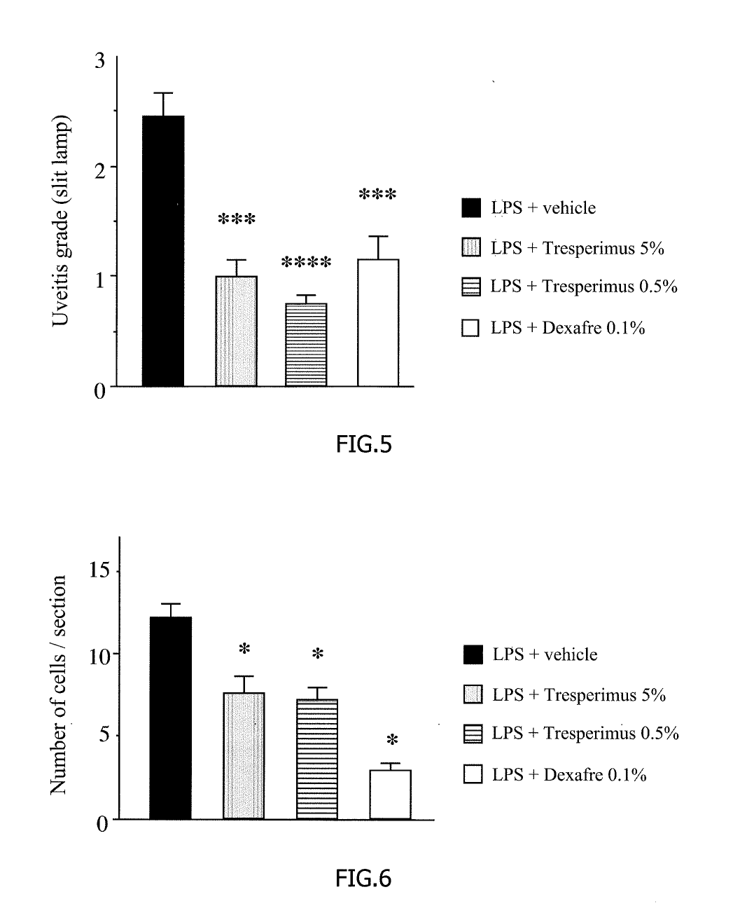

To confirm the clinical effect observed with tresperimus, the total number of cells present in the anterior chamber of the eye was counted and it clearly appears, as shown in FIG. 6, that the infiltration of inflammatory cells was significantly reduced (average number of cells/section: 7.7±0.9, n=13 sections, p=0.003 and 7.3±0.7, n=13 sections, p=0.0004) compared to control animals (average number of cells/section: 12.2±0.8, n=17 sections).

These results illustrate the fact that the instillation of tresperimus in the eye of a rat allowed beneficial effect to be obtained with a reduction of the ocular inflammation in a model of endotoxin-induced uveitis. The results suggest that tresperimus makes it possible to treat the clinical signs of uveitis by instillation of eye drops and more generally to treat severe conjunctivitis since as of today these pathologies are mainly treated by corticoids and immunosuppressants active in these 2 animal pharmacological models.

### Example 2

### Dry Eye Syndrome

Current therapies are essentially palliative and aim at replacing or maintaining a subject's tears by the frequent application of artificial tears. Severe dry eye, characterized by severe corneal damage with an increased risk of secondary infections can occasionally be treated by an anti-inflammatory therapy.

Several animal models have been developed to reflect the different pathophysiological mechanisms involved in KCS. The effect of tresperimus was studied in a mouse model of dry eye using the pharmacological inhibition of tear production which induces epithelial changes of the ocular surface resembling human KCS, which changes are exacerbated by a desiccating environmental stress.

Dry eye is induced in mice by the combination of scopolamine, which blocks the muscarinic cholinergic receptors of lacrimal glands, and by placing the mice in an extractor hood that reduces humidity and increases air flow. The production and volume of aqueous tears, tear clearance, and the corneal barrier function are evaluated before treatment, and then twice a week after treatment. The results are compared between groups of untreated control mice, and groups of mice placed in the extractor hood, treated with the anticholinergic agent scopolamine, treated or not with tresperimus.

This model of experimentally induced dry eye leads to epithelial changes of the ocular surface, with corneal fluorescein staining, to an altered corneal epithelial barrier function, to a reduced density of conjunctival goblet cells, and to an increased conjunctival epithelial proliferation. This animal model mimics the aqueous-deficient and evaporative components of human dry eye syndrome.

### Materials and Methods

I. Induction of Dry Eye with Cholinergic Receptor Blockade and Desiccation in an Extractor Hood

Male 129SV/CD-1 mice were used in this study and received three sub-cutaneous injections of 200 μl of scopolamine at 2.5 mg/mL in saline for 21 days. The mice were placed in an extractor hood (humidity <50%) during the whole experiment.

### II. Aqueous Tear Production

Tear production (PRTT) was measured with cotton threads impregnated with Phenol Red (Zone-quick; Menicon, Japan) applied to the ocular surface in the lateral canthus for 60 seconds. Wetting of the thread was measured in millimeters, using the scale on the cotton thread.

### III. Stability of Tear Film

The stability test of the tear film (TBUT) is used to evaluate the eye dryness by measuring the time that elapses between a full wink and the development of the first sign of a dry spot on the tear film.

One microliter of 0.1% sodium fluorescein was applied to the conjunctival bag and the time (in seconds) after which a dry spot appears was measured after three winks. 90 s later, the damage to the corneal epithelium was measured and photographed with a slit lamp biomicroscope using a cobalt blue light. A clinical score was drawn up using the Draize scoring scale.

### Results

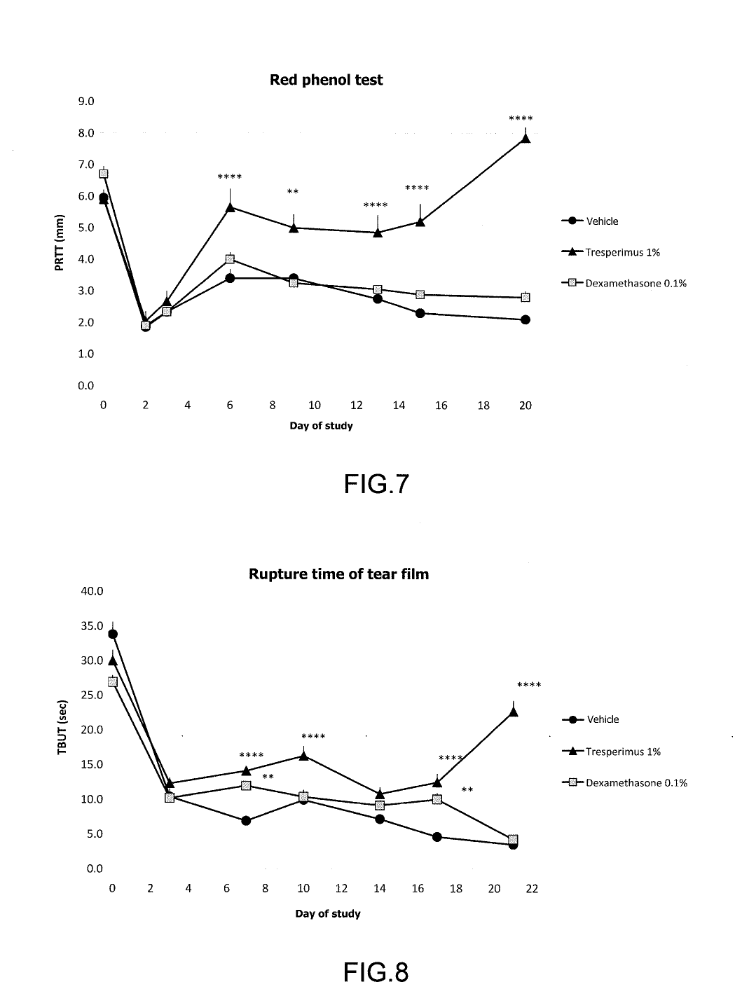

The tear volume was measured during three weeks in C57B16 mice using the Red Phenol test. The results reported on FIG. 7 are expressed as the average tear volume (in millimeters) ±standard error of the mean (SEM). They show that the tear volume dramatically decreased two days after sub-cutaneous injections of scopolamine. Instillations of tresperimus twice a day at the dose of 1% (m/m) in a 0.1% solution of sodium hyaluronate in aqueous saline (0.6% NaCl), greatly improved the tear volume from day 6 to day 20 compared to mice treated with a vehicle made up of a 0.1% solution of sodium hyaluronate in aqueous saline (0.9% NaCl) (two-factor variance analysis using the Bonferroni multiple comparison test, p<0.0001). In contrast, instillations of 0.1% dexamethasone twice a day showed no significant effect on the tear volume.

FIG. 8 shows that treatment with scopolamine and an exposure to desiccated air led to a decrease in the stability of the tear film, as measured by the tear film rupture test, with an important decrease the first 3 days then a progressive decrease until day 21. The administration of tresperimus by 1% instillations twice a day significantly improved the stability of the tear film from day 7 to day 21 compared to mice treated with the vehicle made up of a 0.1% solution of sodium hyaluronate in aqueous saline (0.9% NaCl) (p<0.0001); by contrast dexamethasone only showed a modest effect which did not continue on day 21 In conclusion these results showed that a topical application of tresperimus has beneficial effects on dry eye syndrome by increasing tear secretion and the stability of the tear film, which are two characteristic clinical parameters of dry eye. These results prove the interest of tresperimus instillations for the treatment of clinical signs of dry eye.

### Example 3

### Diabetic Retinopathy

Laser photocoagulation is still the standard of care treatment, and vitrectomy is used in case of retinal detachment. However a significant proportion of subjects is refractory to laser photocoagulation, and with time, retinal pigment epithelium atrophy associated with the laser scars occasionally progresses under the fovea causing decreased vision. Ranibizumab was recently approved for the treatment of macular edema but other anti-VEGF agents (bevamizubab) are used off label. A combined treatment with anti-VEGF agents could delay laser treatment. Corticosteroids make it possible to notice a regression of macular edema and neovascularization. However, adverse effects are frequent (ocular hypertension, cataract, endophtalmitis); moreover, long-term efficacy in diabetic macular edema has not been demonstrated compared to laser therapy.

The effect of tresperimus has been evaluated in rats using a commonly described model of diabetic retinopathy, the streptozotocin-induced type I diabetes model. This rat model mimics the human disease by inducing hyperglycemia associated to the destruction of the beta-cells of the pancreas, which cells normally regulate glycaemia by producing the hormone insulin. Although there are vascular changes in this model, the vasculopathy does not progress to neovascularization as observed in humans.

Streptozotocin is intravenously injected to fasted rats. Hyperglycemia rapidly develops over five days following the streptozotocin treatment. Three weeks after the induction of diabetes, the levels of VEGF and inflammatory biomarkers are determined in the vitreous body. Electroretinogram (ERG) measurements of a- and b-wave as well as oscillatory potentials are analyzed to monitor photoreceptor damages. The results are compared between the control group of non-diabetic rats and the group of diabetic rats treated with tresperimus or a vehicle.

### Materials and Methods

### I. Induction of Diabetes by Streptozotocin

Diabetes was induced in Sprague Dawley (SD) rats (200 g) after overnight fasting by a single 60 mg/kg intravenous injection of streptozotocin (Sigma) in sodium citrate buffer, pH 4.5. Control non diabetic animals received citrate buffer only. Five days later, animals with a glycemia above 5 g/L were considered diabetic.

1. Inflammation Biomarkers

Three weeks after the induction of diabetes by streptozotocin, the eyes of the rats were excised and the vitreous bodies were isolated. Several inflammation biomarkers were measured using a multiplex Luminex assay kit for rats (VEGF, MCP-1, ICAM-1, IL-6, IL-1beta; Procarta) according to the manufacturer's recommendations.

2. Electroretinoqraphy (ERG)

Diabetic rats were adapted to darkness overnight before ERG examination using an electroretinograph from the company LKC. A series of dark-adapted intensity responses was recorded using a series of Ganzfeld flashes to obtain rod-mediated retinal responses. The amplitude and latency of the individual ERG waveform components (a- and b-waves, flickers) and the oscillatory potentials were measured conventionally.

### II. Results

The effect of tresperimus was evaluated in the experimental model of diabetic retinopathy induced by streptozocin in SD rats. Streptozocin destroyed the beta cells of the pancreas and induced a hyperglycemia, thus mimicking type 1 diabetes. The retina of diabetic animals showed biochemical and electrophysiological abnormalities correlated to inflammation.

Instillations of tresperimus administered twice a day for two weeks at the dose of 0.2% (m/m) in a 0.1% solution of sodium hyaluronate in aqueous saline (0.9% NaCl), did not modify glycaemia or body weight compared to diabetic rats treated with a vehicle made up of a 0.1% solution of sodium hyaluronate in aqueous saline (0.9% NaCl).

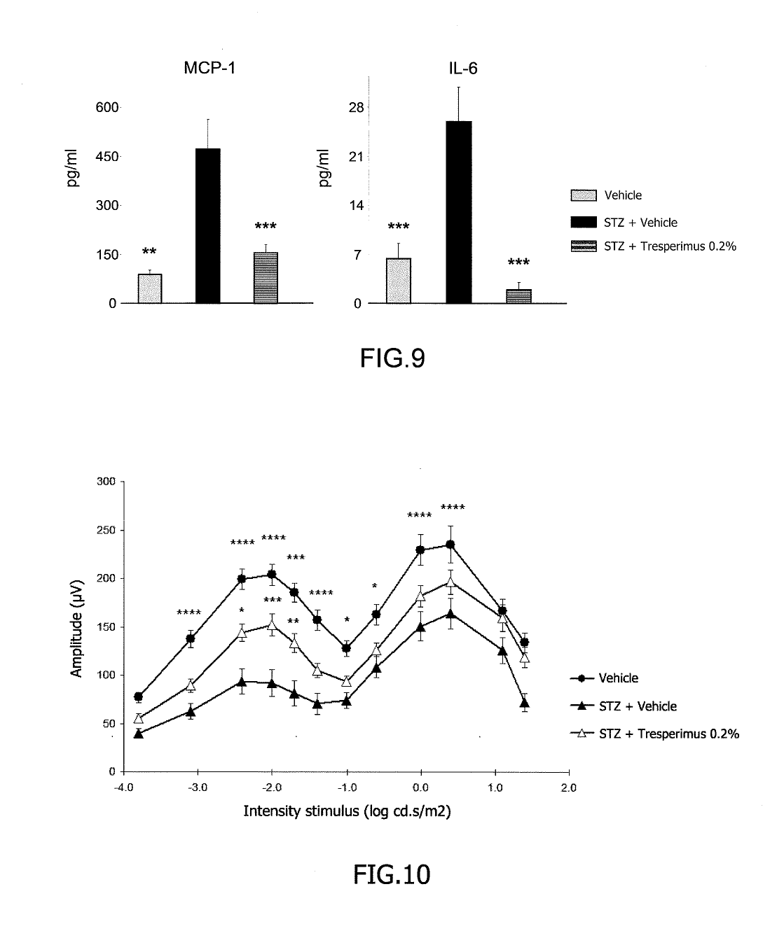

Cytokine and chemokine levels were evaluated on samples of vitreous body using the multiplex Luminex assay technology. The results reported on FIG. 9 show that MCP-1 and IL-6 levels in the vitreous medium dramatically increased three weeks after the induction of diabetes by streptozocin. A two-week treatment with instillations of tresperimus at a dose of 0.2% twice a day in both eyes from day 7 to day 21, significantly reduced MCP-1 and IL-6 levels in the vitreous body of diabetic rats, suggesting an inhibiting effect on monocyte recruitment during the inflammatory process (one-factor variance analysis using the Dunnett multiple comparison test, p≦0.001).

Three weeks after the streptozocin treatment, the ERG examination revealed that diabetic rats showed after adapting to darkness a decrease in the amplitude of the a- and b-waves, an abnormality of the oscillatory potentials, and a large deterioration of the flickers (FIG. 10) irrespective of the intensity of the light flash. The cones and rods are the two types of photoreceptors affected by hyperglycemia. FIG. 10 shows that after two weeks of treatment with twice a day administration of ocular instillations of 0.2% tresperimus, tresperimus significantly improved the amplitude of the flickers compared to the control batch (diabetic rats treated with vehicle), and also improved the amplitude of the a- and b-waves and of the oscillatory potentials, suggesting a neuroprotective effect of the retinal functions, notably the cones and rods in diabetic rats.

In conclusion these results thus show that the topical administration of tresperimus has beneficial effects on the retina of diabetic rats by decreasing the retinal inflammation level and by protecting the neuro-retinal functions, notably the cones and rods, from hyperglycemia. These results thus prove the interest of tresperimus instillations for the treatment of diabetic retinopathy in man and for the prevention of vision impairment in diabetic subjects.

1.-25. (canceled)

26. A method of treating or preventing an ocular inflammatory disease, which comprises administering to a subject in need thereof a therapeutically effective amount of a compound of formula (I) or of a pharmaceutically acceptable salt thereof: 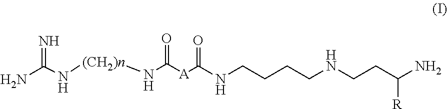
in which:
n is equal to 6 or 8,
A is a bond, a group CH₂, a group CH(OH), a group CHF, a group CH(OCH₃), a group CH₂NH or a group CH₂O,
R is H or CH₃.

27. The method according to claim 26, wherein the compound of formula (I) is N-[4-[(3-aminopropyl)amino]butyl]-carbamic acid, 2-[[6-[(aminoiminomethyl)amino]-hexyl]amino]-2-oxoethyl ester or a pharmaceutically acceptable salt thereof.

28. The method according to claim 27, wherein the compound of formula (I) is N-[4-[(3-aminopropyl)amino]butyl]-carbamic acid, 2-[[6-[(aminoiminomethyl)amino]-hexyl]amino]-2-oxoethyl ester, tri-hydrochloride.

29. The method according to claim 26, wherein the compound of formula (I) is N-[4-[(3-aminobutyl)amino]butyl]-carbamic acid, 2-[[6-[(aminoiminomethyl)-amino]hexyl]amino]-2-oxoethyl ester or a pharmaceutically acceptable salt thereof.

30. The method according to claim 29, wherein the compound of formula (I) is N-[4-[(3-aminobutyl)amino]butyl]-carbamic acid, 2-[[6-[(aminoiminomethyl)-amino]hexyl]amino]-2-oxoethyl ester, tetra-hydrochloride.

31. The method according to claim 26, wherein the ocular inflammatory disease is non-infectious uveitis, severe conjunctivitis, dry eye syndrome or diabetic retinopathy.

32. The method according to claim 31, wherein the ocular inflammatory disease is dry eye syndrome.

33. The method according to claim 31, wherein the severe conjunctivitis is vernal keratoconjunctivitis.

34. The method according to claim 26, wherein the compound of formula (I) or pharmaceutically acceptable salt thereof, is administered as eye drops.

35. The method according to claim 26, wherein the compound of formula (I) or pharmaceutically acceptable salt thereof, is administered as an injectable or an implantable system.

36. The method according to claim 26, wherein the compound of formula (I) or pharmaceutically acceptable salt thereof is administered in combination with an anti-VGEF agent, an anti-TNF agent, a corticosteroid, a non-steroidal anti-inflammatory agent, an antibiotic or an immunosuppressant.

37. The method according to claim 36, wherein the administration of the compound of formula (I) and of the other agent is simultaneous, sequential or over a period of time.

38. An aqueous topical formulation which comprises a compound of formula (I) or a pharmaceutically acceptable salt thereof as sole active substance, and one or more pharmaceutically acceptable excipient(s) suitable for topical administration, wherein the concentration of the active substance is from 0.001% to 1.5%: 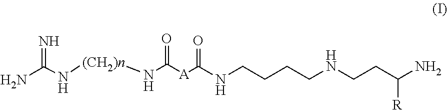
in which:
n is equal to 6 or 8,
A is a bond, a group CH₂, a group CH(OH), a group CHF, a group CH(OCH₃), a group CH₂NH or a group CH₂O,
R is H or CH₃.

39. The aqueous topical formulation according to claim 38, wherein the concentration of the active substance is from 0.01% to 1.5%:

40. The aqueous topical formulation according to claim 38, which is in the form of eye drops having a substantially neutral pH.

41. The aqueous topical formulation according to claim 40, which comprises an excipient selected from: hyaluronic acid or a derivative thereof; one of sodium chloride and glycerol; and mixtures thereof.

42. An aqueous injectable formulation which comprises a compound of formula (I) or a pharmaceutically acceptable salt thereof as sole active substance, and one or more pharmaceutically acceptable excipient(s) suitable for an injectable administration in the eye, wherein the concentration of the active substance is from 0.1 μM to 100 mM: 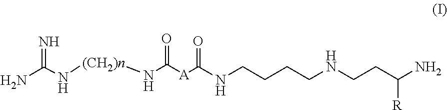
in which:
n is equal to 6 or 8,
A is a bond, a group CH₂, a group CH(OH), a group CHF, a group CH(OCH₃), a group CH₂NH or a group CH₂O,
R is H or CH₃.

43. The aqueous injectable formulation according to claim 42, wherein the concentration of the active substance is from 1 μM to 10 mM.

44. The aqueous injectable formulation according to claim 42, which is an intraocular or a periocular formulation.

45. The aqueous injectable formulation according to claim 42, which is in the form of an intravitreal injectable solution having a substantially neutral pH.

46. The aqueous injectable formulation according to claim 45, which comprises sodium chloride or glycerol.

47. The aqueous injectable formulation according to claim 44, which is an implant.
